(19) United States
(12) Patent Application Publication
(10) Pub. No.: US 2026/0096713 A1
(43) Pub. Date: Apr. 9, 2026

(54) SURFACE SANITIZING APPARATUS

(71) Applicant: Richard Mark Capehart, Richmond, VA (US)

(72) Inventor: Richard Mark Capehart, Richmond, VA (US)

(21) Appl. No.: 19/343,593

(22) Filed: Sep. 29, 2025

Related U.S. Application Data
(60) Provisional application No. 63/703,234, filed on Oct. 4, 2024.

(51) Int. Cl. A47L 13/26; A47L 13/11; A47L 13/12; A47L 13/42; A61L 2/18; A61L 2/26; F21S 9/03; F21V 33/00

(52) U.S. Cl. CPC A47L 13/26; A47L 13/11; A47L 13/12; A47L 13/42; A61L 2/18; A61L 2/26; F21S 9/03; F21V 33/0064; A61L 2202/15; A61L 2202/16; A61L 2202/17

(57) ABSTRACT

A sanitizing apparatus includes an outer housing, an inner container, and a sanitizing bottle. The outer housing has an exterior surface containing an access window and the inner container is arranged within the outer housing. The inner container has an opening that aligns with the outer housing access window. At least one of the outer housing and inner container are rotatable relative the other to align the inner container opening and outer housing access window. The sanitizing bottle is then arranged within the inner container and can be accessed to clean a surface. Once cleaned, the bottle is placed back into the inner container and the outer housing or inner container is rotated to secure the bottle therein. Preferably, the sanitizing bottle has one of a scrubber-equipped flip-top and twist-off cap.

### BACKGROUND

The present disclosure relates to a surface sanitizer, and more particularly a modular sanitizing apparatus.

Surface sanitizing apparatus are well-known. These include squirt bottles, sponges, brushes, and other devices for applying a substance and scrubbing a surface. Such known products are typically stored in a standard storage area, such as a cabinet, drawer, center console or similar area. When needed, a user must first retrieve the product from where it is stored, use the product and then return it to its storage place.

With increased demand for public and home sanitation—especially in high-traffic outdoor areas—there is a growing need for improved sanitizing devices. Traditional sanitizing dispensers often lack visual appeal, weather resistance, and the convenience needed site-specific installation. This renders them impractical for everyday use on surfaces such as mailboxes, decks, patios, patio furniture, and vehicles.

At the same time, while solar-powered lighting fixtures and decorative address markers are common, they serve purely aesthetic functions and offer no sanitizing capabilities. Likewise, conventional cleaning tools are often stored indoors and inconvenient to access when needed.

There is thus a need for a cleaning device that includes more accessible storage of a cleaning product for better access and ease of use.

### SUMMARY OF THE INVENTION

Accordingly, it is an object of the present disclosure to provide a sanitizing apparatus that includes an outer housing, an inner container, and a sanitizing bottle. The outer housing has an exterior surface containing an access window and the inner container is arranged within the outer housing. The inner container has an opening that aligns with the outer housing access window. At least one of the outer housing and inner container are rotatable relative the other to align the inner container opening and outer housing access window. The sanitizing bottle is then arranged within the inner container and can be accessed to clean a surface. Once cleaned, the bottle is placed back into the inner container and the outer housing or inner container is rotated to secure the bottle therein. Preferably, the sanitizing bottle has one of a scrubber-equipped flip-top and twist-off cap.

In one embodiment, the sanitizing apparatus further includes a solar lighting module mounted at an upper portion of the outer housing configured to provide illumination, along with a modular base connected with a lower portion of the outer housing to maintain the outer housing in an upright position.

In a second embodiment, the outer housing is constructed from a material selected from the group consisting of plastic, thin sheet metal, or 3D-printed polymer.

In another embodiment, the modular base includes an interchangeable mount including at least one of a pedestal, ground stake, and shepherd's hook attachment.

In yet another embodiment, the solar lighting module includes a solar panel, rechargeable battery, and LED circuit.

In a further embodiment, the apparatus also includes a drainage port arranged at the base of the outer housing for water resistance and a fragrance insert positioned within the outer housing to emit scent during use.

A further embodiment still, the scrubber cap includes an integrated squeegee and the bottle is refillable.

In an additional embodiment, the inner housing contains further openings and the solar lighting module is configured to project light through these openings to create illuminated visual effects.

It is further an object of the present disclosure to provide a method of sanitizing outdoor surfaces using the above-described apparatus. The method includes rotating the inner container housed within the outer housing to align the inner container opening with the corresponding outer housing access window, removing or exposing the bottle, dispensing a sanitizing fluid onto a target surface by actuating the squeeze bottle and scrubbing the target surface using the integrated cap to remove contaminants, and placing the bottle back into the inner container opening and rotating the opening to secure the bottle within the outer housing.

In one embodiment of the method, the additional step of mounting the apparatus on a surface including one of a mailbox post, patio deck, table surface, shepherd's hook, and ground stake is included.

In another embodiment, the steps further include the step of illuminating an address display or decorative design on the outer housing using the integrated solar lighting.

### BRIEF DESCRIPTION OF THE DRAWINGS

FIGS. 1 and 2 are perspective views of a sanitizing apparatus according to the present disclosure;

FIGS. 3 and 4 are front and side views of the apparatus of FIGS. 1 and 2;

FIG. 5 is a perspective view of the apparatus from FIG. 1 with the outer and inner housing separated;

FIGS. 6 and 7 are perspective views of a sanitizing bottle of a sanitizing apparatus according to the present disclosure;

FIGS. 8-11 are embodiments of sanitizing bottle tops of a sanitizing bottle according to the present disclosure;

FIGS. 12-14 are bottom views of a modular base of a sanitizing apparatus according to the present disclosure;

FIGS. 15-17 are perspective views of an embodiment of a sanitizing apparatus with a lighting module according to the present disclosure;

FIG. 18 is a further embodiment of a sanitizing apparatus with decorative elements according to the present disclosure;

FIGS. 19 and 20 are top views of a solar module for a sanitizing apparatus according to the present disclosure; and

FIGS. 21-24 show a supportive stand for a sanitizing apparatus according to the present disclosure.

### DETAILED DESCRIPTION

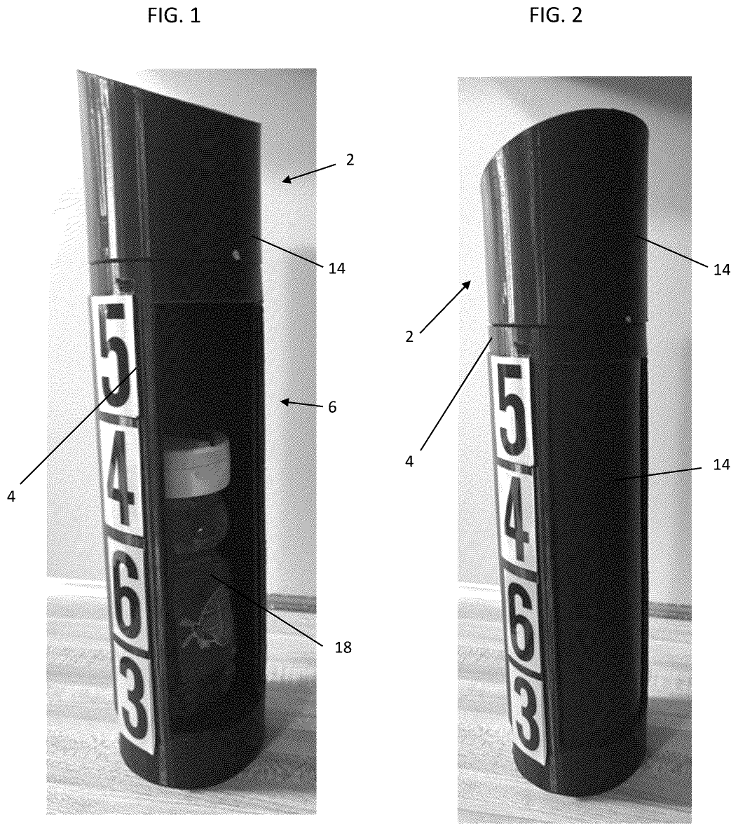

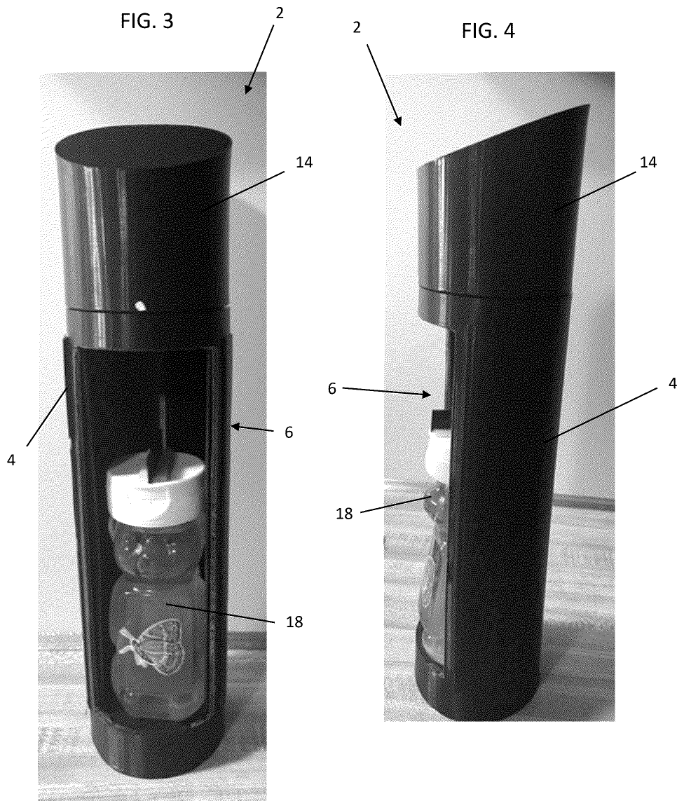

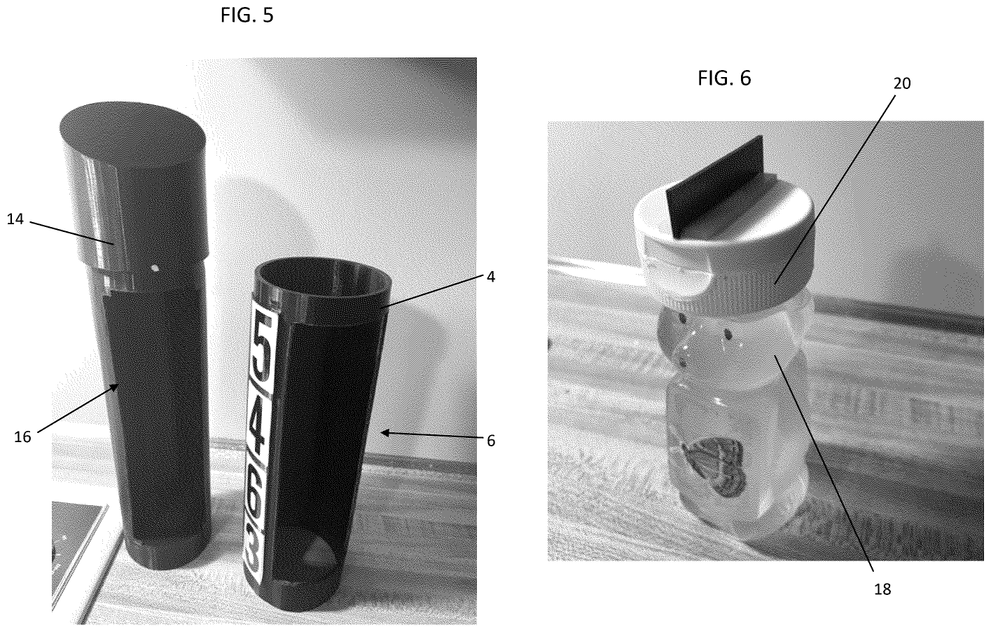

Referring first to FIGS. 1-5, the supportive protective stand 2 for this product includes an outer oblong round metal tube 4, twelve inches high and 3.15 inches wide that stands upright. There is an oblong cut 6 in the front of this tube measuring nine inches in height and two and a half inches in width, which allows access to the cleaning solution as well as protection and anonymity for the product. The oblong cut measures approximately one inch from the top of the tube and two inches from the bottom of the tube.

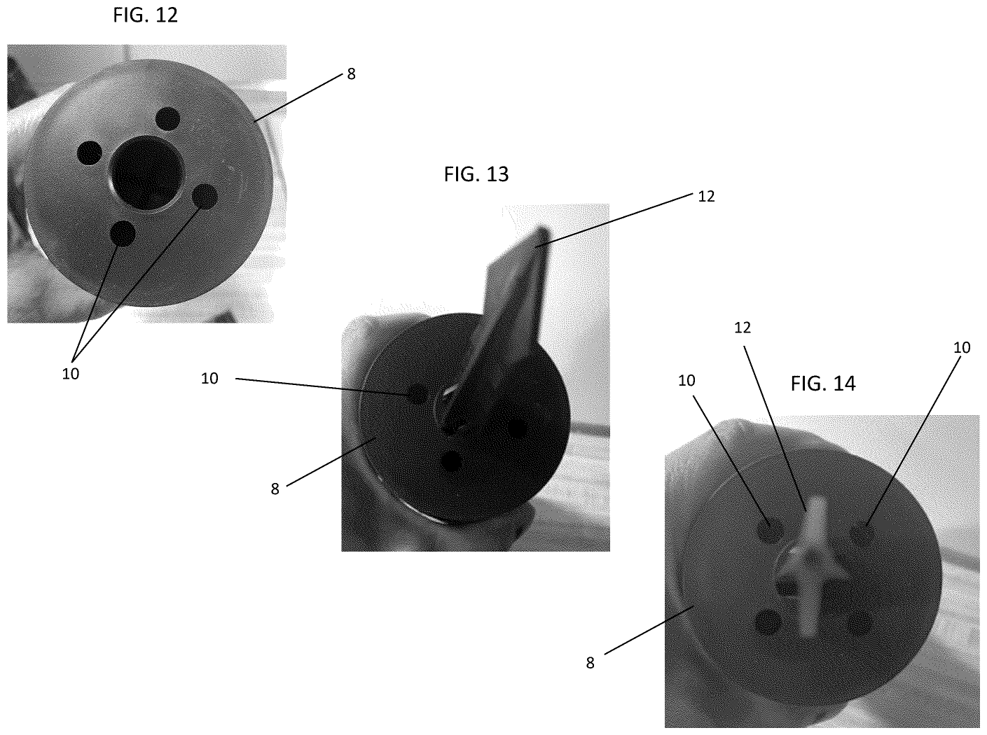

Referring now to FIGS. 12-14, a supporting base 8 is glued to the inside of the tube 4 at the bottom and includes water drain holes 10 in the bottom of the base. In the middle of the base is a protrusion that supports a plastic grounding six-inch long spike 12 with a point which inserts into the ground and is capable of securing the product to the ground. A second oblong tube 14, similar to the first one, which is made of metal and has a supporting base of its own, also has an oblong cut 16 in the front that lines up with the cut in the first tube 4. This inner tube slides into the outer supportive tube, to form a rotatable cylinder, and the two cuts 6, 16 in the two metal pieces, line up in the front. The inner tube 14 supports and houses the container 18 for the cleaning sanitizing solution.

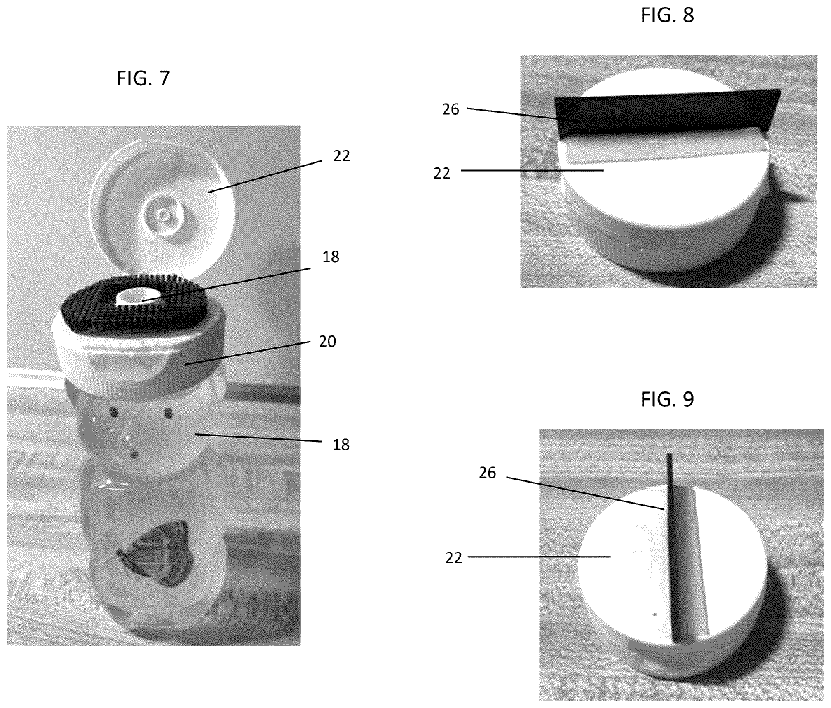

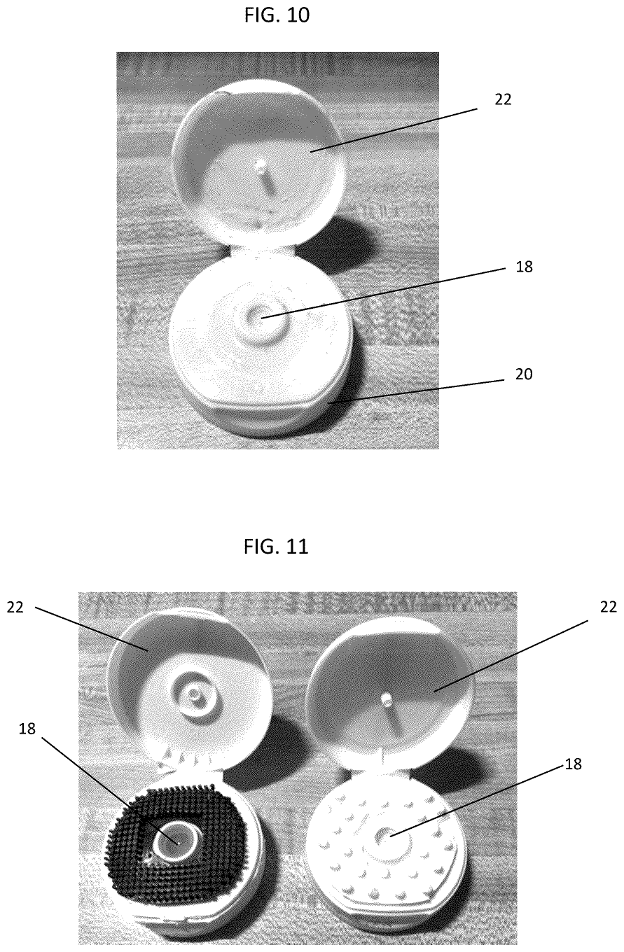

Referring to FIGS. 6-11, the liquid cleaning/sanitizing container 18, which is made of plastic, supports a screw on plastic cap 20 with an attached cap cover 22. Attached to the cap is a rubber, plastic, or silicone material, with short rubber, plastic or silicone ridges, containing a hole in the middle which is used for wiping away or scrubbing debris, on a dirty or unsanitary surface. This piece is glued on top of the inner cap and rest underneath the outer cap cover. Protruding through this piece is a supporting leakless dispenser cap 24, which exposes a structural weakness allowing cleaning and sanitizing solution to be squirted onto a surface to be cleaned, by squeezing the supporting container in the center.

The scrubbing piece between the inner and outer cap is used for scrubbing away unsightly debris on a structure. The cleaning and disinfecting solution in the container, is used to clean and disinfect dirty surfaces. On the very top of the cap is a secured squeegee 26, supported by two pieces of plastic, which is glued on the top of the cap cover. This part of the product is used to wipe away any excess cleaning solution after the surface has been cleaned and sanitized. Refills for the container, containing cleaning and sanitizing solution can be purchased jointly with the entire invention or separately every three to six months as needed. These refilled bottles may be used to support advertising and decorative themes.

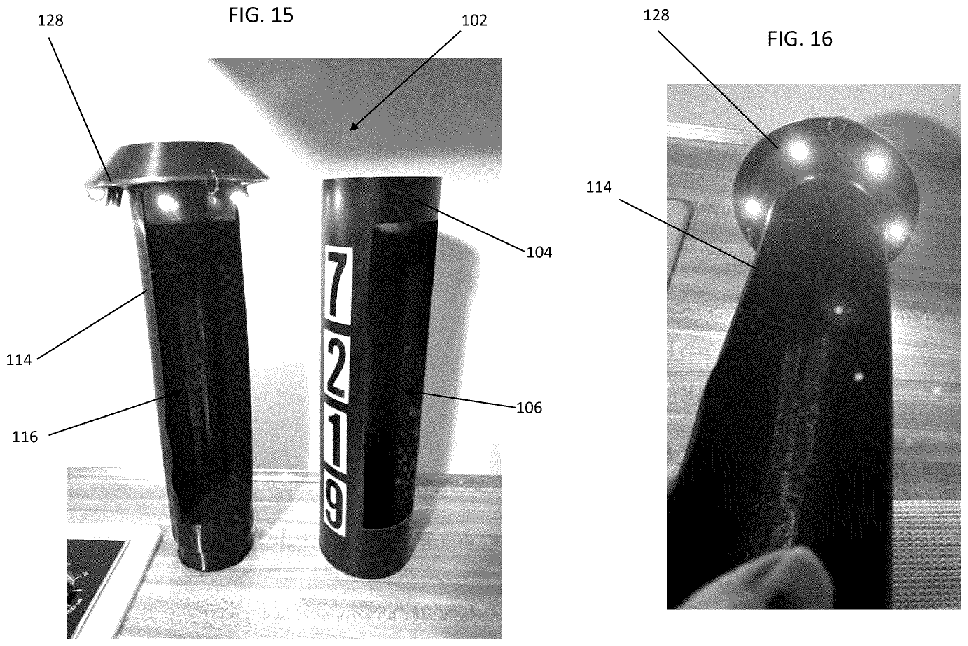

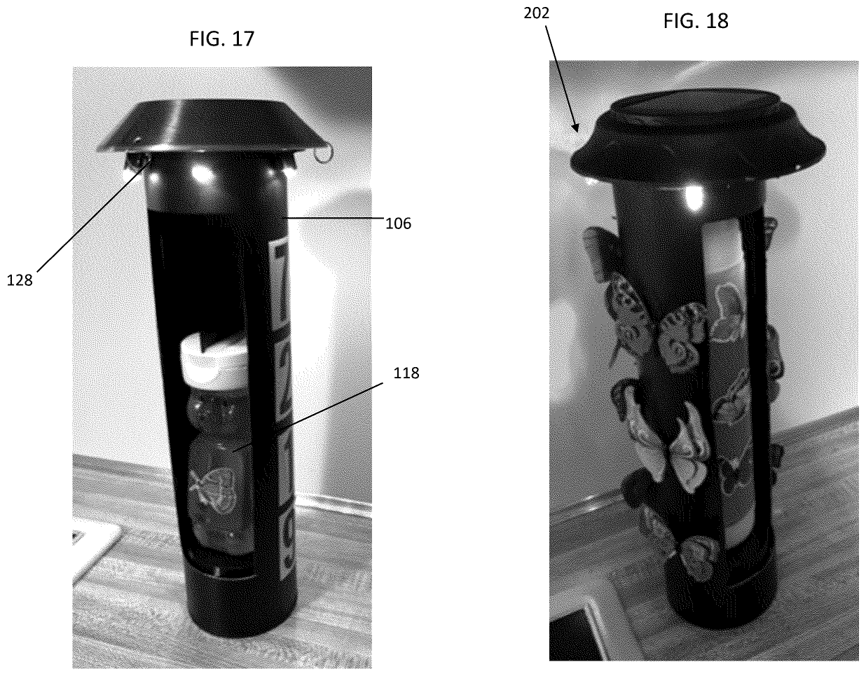

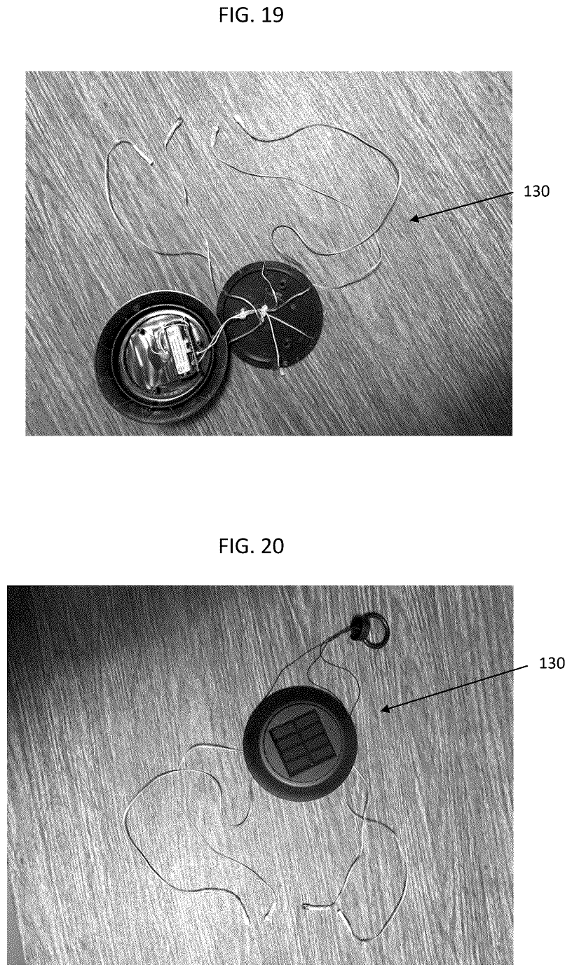

As shown in FIGS. 15-17, one embodiment 102 includes a solar light 128 that is attached to the inner tube by using a round hollow connector with a flat lip at the top that is glued to the inside of the tube, and to the solar light as well. The solar lights extend beyond the two tubes and are angled through supporting holes in the solar light in order to give maximum light support, so as to light up the outside of the product which may project reflective address numbers, decorative magnets, advertising, etc. FIGS. 19 and 20 show a solar module 130 for the solar light.

A spin of the solar top reveals the cleaning solution 118, and another spin of the solar top will enclose the cleaning solution and hide it from view.

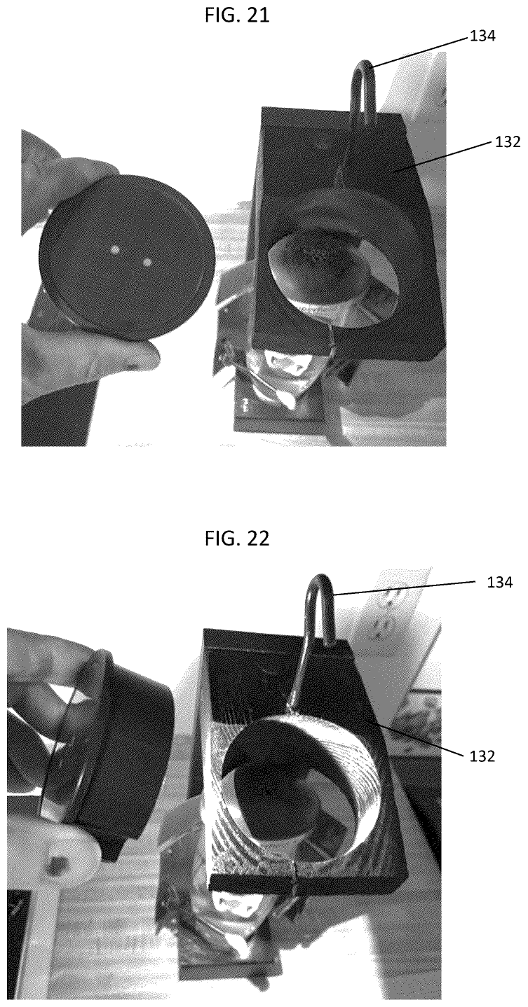

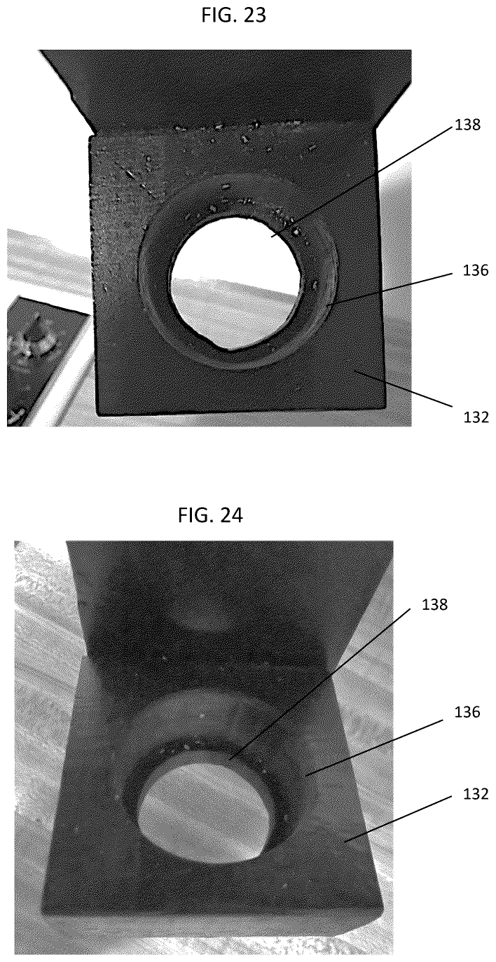

Referring now to FIGS. 21-24, there is an additional supportive stand 132 made of wood which measures approximately twelve inches in height and three-and-a-half inches in width. This can be decorated, painted, and solar lit, and hung on a shepherd's hook with an eye hook or similar hook 134. This piece can be nailed together with wood or produced in a plastic form. There is a supportive hole 136 in the bottom of this stand that supports the cleaning container with an additional smaller hole 138 inside of it, to keep the bottle from sliding through.

Yet another version is a stand that is manufactured by a 3-D printer and made of a plastic substance, operating in the same way the metal stand operates.

The refill bottle of this product can be kept in the trunk of a car and used to eliminate bird fecal debris and any other messy debris that may incur.

This entire product is used to clean and sanitize unsightly surfaces with an emphasis on mailboxes, patio furniture, decks, and cars that are consistently infiltrated by bird debris and other messes. This product can be used in conjunction with advertising promotions, and or themes, as well as cartoon, personality, or character identification. This product, both the sanitizing cleaner and the housing, can be produced in the form of cartoon characters, people, or specific objects.

This product works by anchoring it directly into the ground, near a mailbox, sitting it on a table or directly on your deck floor as a nighttime guiding light. It can hang on a shepherd's hook or be effective as a refill bottle that goes in the trunk of your car, that can eliminate bird and other debris from your car.

As noted previously, to make use of the product, spin the solar cap to reveal the cleaning/sanitizing solution. Once revealed, pop open the top and quirt the sanitizer on the affected area, scrub away the debris, rinse away the debris, clean the top of the bottle first on the grass and then on the back of a firm surface, rinse the surface with cleaning sanitizer, close the top of the bottle, and use the squeegee to wipe away any excess cleaner. Then store the product away for future use.

In one embodiment as described above, there is a cap with an attached covering cap, such as one used with a Heinz 57 or American ketchup top, in the closed position. On top of the cap are two flat single plastic strips one-and-a-half to two inches long and a quarter inch wide, glued to the top. These two strips support a flat piece of rubber one-and-a-half to two inches long, and a quarter inch high, which slides between these two plastic pieces on either side and works like a squeegee, used to wipe away excess liquid cleaner, from a firm surface such as a mailbox, deck, patio, furniture, or car.

When the outer cap is open and the inner cap is exposed, you will note a protruding tip in the center of the cap with a structural weakness that allows cleaner to pass through the top when a supporting liquid bottle is attached to the cap and squeezed in the center.

The structural plastic weakness in the protruding tip is designed to keep the product from leaking, until the plastic supporting bottle filled with liquid, is squeezed from the center. The protruding tip on top of the inner cap is surrounded by a raised piece of plastic or rubber with raised ridges designed to scrub away debris on firm surfaces, such as mailboxes, decks, patio furniture, or cars. This plastic or rubber piece with the raised ridges, is glued on top of the inner cap with the protruding tip coming through the center, at the approximate, or same height, as the scrubbing apparatus. Note the two different designs of the two rubbing apparatuses on the two different tops, the Heinz 57 ketchup top and the American ketchup top.

There is also a bottle that can be used with the device, for instance a twelve-ounce honey bottle that is empty. Remove its top along with any stickers on it. A honey bottle is preferred because it is in the shape of a cute little honey bear and is able to support a ketchup top that fits on the bottle, which is essential to dispensing liquid sanitizer and cleaner when squeezing the bottle in the center, and not leaking when it is not in use. Those with skill in the art will understand that any bottle which can support a top as described herein could be used.

The labels are removed and waterproof butterfly stickers are put in their place for decoration. A taller bottle, for instance a sixteen-ounce bottle approximately nine inches tall, is preferred so long as it can fit the top described herein. This particular bottle is approximately two-and-a-half inches wide and approximately six-and-a-half inches high.

A sanitizing/cleansing solution is added to the bottle. Two preferred cleaning/sanitizing tablets to be considered are DAZZ all-purpose cleaning tablets and Steramine sanitizing tablets, as well as an antifreeze tablet dissolved in sixteen-ounces of water. After adding this cleaning/sanitizing solution, the cap that holds the scrubbing apparatus and the squeegee for wiping away excess cleaner from a firm surface is attached. This is used to clean surfaces such as mailboxes, decks, patio furniture, and cars. This is the final element of the bottle of cleaning/sanitizing solution that can be sold together with the kit or sold separately as a replacement bottle.

As previously disclosed, there is also a housing for the sanitizing/cleaning bottle, which preferably includes a 3-D printed plastic outer cylinder ten inches tall and three inches wide, which includes a hollow round cylinder with an oblong shape that stands straight up and exposes a rectangular cut in the front. This cut in the front for the embodiment shown in the figures is approximately two-and-a-half inches wide, and approximately nine inches in length. It will be understood by those with skill in the art that alternative cuts could be used depending on the size of the cleaning bottle. This outer cylinder may support address identifying numbers on opposite sides.

In addition, this outer cylinder will also house an inner cylinder. This inner 3-D printed cylinder will have a self-supporting bottom and will measure approximately ten inches in height from the bottom, moving up to the top, where it will then spread or fan out about an eighth of an inch to form an easy to grip top with a slant at the top that fits in the outer cylinder, and joins the outer cylinder at the top for a neat, sleek fit. This top will measure three inches in height and the total height of this housing stand will measure thirteen inches tall. Again, the dimensions of this element may change depending on the dimensions of other elements described herein. A twist of the top will turn the inner cylinder freely.

The inner cylinder will include a rectangular cut in the front approximately nine inches long and two-and-a-half inches in width that lines up proportionately to the cut in the outer cylinder which houses the inner cylinder. The spin of this top will either expose or enclose a sanitizing cleaning bottle that it houses. Different size and shapes of cuts are contemplated for different embodiments of this invention.

The bottom of the outer cylinder will house a base at the bottom which will be inserted into and connected with, preferably glued into, the bottom of the outside cylinder. The inserted base will have four water drain holes, as well as an indented round, area that will allow a round spike to be inserted up to a certain stopping point in the base, and a pointed end in the spike, which will allow the spike to be driven into the ground; thereby, securing the product to the ground when needed.

The second housing cylinder is similar to the first housing cylinder, in shape and size. This cylinder, however, is made of a metal substance and carries a solar recharging light at the top, with a battery included inside the solar light to aid with recharging so that it may light up in the darkness.

The solar light has approximately six holes drilled into the bottom of the solar light, which allows six protruding individual lights that extend over the outside of the outer cylinder, which are slightly angled inwardly toward the outer cylinder, to allow for maximum light exposure to the outside of the outer cylinder.

This outer cylinder is an oblong round hollow tube that measures twelve inches long. The front of this outer cylinder has a rectangular cut that begins 1 inch from the top of the tube and continues down the tube nine inches and stops two inches from the bottom. This cut measures two-and-a-half inches wide. The many dimensions and shapes noted above may be altered without deviating from the spirit of the invention.

The bottom end of the outer cylinder has a supporting bottom, which is glued to the inside of this outer cylinder. This supporting bottom will have four holes at the bottom to allow water to drain out. In addition to this will be an indention in the middle of this supportive bottom that will allow the round end of the spike to only be inserted to a certain depth to where the spike will encounter a stopping barrier. The other end of this spike will have a point which will spear into the ground so that this product can be anchored to the ground sufficiently. The cylinder will also have the flexibility of sitting or resting on a flat surface without the spike, such as on a deck, step, or table. The spike will be round at the top for insertion into the supporting bottom, and measure 6 inches in length in order to provide maximum stabilization for the product, if inserted into the ground.

The outer surface of this cylinder can be used to hold reflective address identifying numbers on opposite sides, as well as magnetic butterflies, or weatherproof stick-on butterflies (FIG. 18), or any other decorative or advertising material.

The outer cylinder of this product will house an inner cylinder, which will consist of a round hollow oblong shaped cylinder with a supporting bottom, which will be used to house a container of sanitizing/cleansing solution. This inner cylinder preferably measures ten-and-a-half inches in length and will exhibit a cut in the front that lines up with the cut in the outer cylinder. This cut will begin at the very top of this container and come all the way down to the floor of the supporting bottom. The width of this cut preferably measures two-and-a-half inches wide. When the open face of the inner cylinder is lined up with the open face, of the outer cylinder, then the sanitizing/cleansing container will be easily accessible for taking out of the housing or putting it back in.

When the inner cylinder is rotated one way, the cleaning/sanitizing container it houses is exposed. When turned again, the cleaning/sanitizing container is enclosed. This inner cylinder will slide easily inside the outer cylinder, so that it is flush up against it, and it will be able to turn freely inside the outer cylinder.

At the top of the inner cylinder will be an attached round hollow ring glued to the inside of the inner cylinder with a small flat lip on top that will extend over the top edge of the outer cylinder and will be connected, for instance glued, inside of the inner cylinder with the flat lip facing upward. The solar light will be glued on to the flat lip creating a completely contained housing for the sanitizing/cleaning solution that the supportive container houses.

Another housing stand for the sanitizing/cleaning container would be a stand made of wood, or plastic sculpted in the same fashion. The wood stand will consist of a stand that measures twelve inches in height and three-and-a-half inches in width. It would consist of a base one-and-a-half inches thick and three-and-a-half inches in length. In the center of the base would be a circular hole approximately two-and-a-quarter inches wide with an extended lip at the bottom of the hole measuring a quarter of an inch which would keep the sanitizing/cleaning solution container that it holds and houses, from falling through the supporting base.

Attached to the base would be an oblong rectangular flat piece of wood measuring twelve inches in length, three-quarters of an inch in thickness, and three-and-a-half inches in width that would line up with the base and would be attached to the base with two screws. This piece would be attached to a piece at the top with two screws that would support a piece of wood measuring five inches in length, one-and-a-half inches in height, and three-and-a-half inches in width. This piece would also have an angle cut that would house a circular solar light three-and-an-eighth inches in diameter, in order that the light may be angled in such a way, as to shine the maximum amount of light to the sanitizing/cleaning solution that it houses. The specific size and shape of the elements described above may vary without deviating from the spirit of the invention.

A hook will be placed behind the solar light so that this product may have the ability to sit on a flat surface or be hung on a shepherd's hook. This stand will hang a container of sanitizing/cleaning solution that will have the capability of being inserted into the base of the housing stand for secure support and storage.

For this embodiment, the cap of the sanitizing/cleaning container will allow the cleaning/sanitizing solution to be stored upside down and allow cleaning/sanitizing solution to be released directly from the dispensing sprout without leaking when squeezing pressure is released from the middle of the sanitizing/cleaning container.

In another embodiment, decorative themes such as waterproof character stickers or prop aids may be added to the housing container, or the sanitizing/cleaning container of liquid solution.

The sanitizing/cleaning container filled with sanitizing/cleaning solution, along with the supporting top that squirts sanitizer on the surface to be cleaned, eradicates debris, and wipes off excess cleaner with the squeegee is a product that can be sold as a refill/replacement product, or purchased separately to be stored in the trunk of a car, and used to eliminate bird debris, and any other debris from the car.

In one embodiment the solar light is battery powered, for instance it may be equipped with an AAA battery, and includes six individual extended wires extending from the power source to six individual LED lightbulbs. The power source may vary in other embodiments. These lightbulbs extend through six holes in the bottom of the solar light and are angled to give maximum light support to the outer cylinder, as well as the sanitizing/cleaning container when the cylinder is in the open position.

The base that goes in the bottom of the outside cylinder and houses the sanitizing/cleaning container, is the same width as the inner cylinder. It measures one-and-a-half inches high and is glued to the inside of the outer cylinder. This base rest freely up against the inner cylinder, allowing the inner cylinder to move freely and turn. In the middle of the outer cylinder base, that is glued to the inside of the outer cylinder, is a circular hole three-quarters to one inch in diameter, and one inch deep with a stopping barrier that has an X cut out on top in the center, to drain any excess liquid that may build up from around the inside cylinder. Around the center hole will be four smaller holes for any excess liquid or water that may need to drain from the cylinder. A six-inch spike three-quarters to one inch in diameter, will go into the supporting base that is glued to the inside of the outer cylinder, and the opposite end of the spike, which will have a point on it, will enable this product to stand on its own when driven into the ground. The stake supporting this cylinder will have ridges around it, so that any excess liquid may drain from the top to the bottom of the stake.

A pair of devices, including cylinder housing, light reflecting, address identifiers with solar light, and containerized sanitizing/cleaning solution, can also be used at the end of driveways to keep vehicles on the driveway and out of ditches as they mark the path of the driveway.

The sanitizing fluid delivery mechanism may be adapted for a pump-action sprayer, gravity-fed nozzle, or pressurized dispenser, and may feature interchangeable scrubber heads such as bristle brushes, microfiber pads, or dual-function squeegee-scrubber tips. The squeeze bottle itself may be configured in various volumes and ergonomics to serve domestic or commercial needs.

The solar lighting module may incorporate motion-sensing, multicolor illumination, or address spotlights. In certain configurations, the solar light may shine downward through cut-out facial or decorative features in the housing to create illuminated silhouettes or patterns-reminiscent of a carved holiday lantern or thematic light display.

In colder climates, the sanitizer fluid may include antifreeze agents, and the housing may feature thermal insulation or removable winter covers. Additionally, indoor-compatible versions of the device may substitute the solar lighting with a standard wall plug or USB-powered LED module, making the invention suitable for kitchen or bathroom sinks, countertops, or utility rooms. These indoor versions may emit ambient lighting through color-shifting or character-themed shades adapted for interior décor.

The unit may also support removable decorative skins or wraps, allowing users to update the theme based on holidays, promotions, or different colors for stylistic variation.

These and other variations are considered within the scope of the invention as defined by the appended claims.

Although the above description includes reference to particular embodiments, it is to be understood that these embodiments are merely illustrative of the principles and applications of the present disclosure. It is therefore to be understood that numerous modifications may be made to the illustrative embodiments and that other arrangements may be devised and employed without departing from the spirit and scope of the present disclosure.

What is claimed is:

1. A sanitizing apparatus comprising:
a. an outer housing having an exterior surface containing an access window;
b. an inner container arranged within the outer housing, the inner container containing an opening alignable with the outer housing access window, wherein at least one of the outer housing and inner container are rotatable relative the other to align the inner container opening and outer housing access window; and
c. a sanitizing bottle arranged within the inner container, the sanitizing bottle having one of a scrubber-equipped flip-top and twist-off cap, whereby when the outer housing is arranged on a surface, one of the outer housing and inner container is rotated to align the access window with the inner container opening, the sanitizing bottle is withdrawn from the inner container to clean a surface, the bottle is placed back into the inner container and the outer housing or inner container is rotated to secure the bottle therein.

2. The sanitizing apparatus of claim 1, and further comprising:
a. a solar lighting module mounted at an upper portion of the outer housing and configured to provide illumination; and
b. a modular base connected with a lower portion of the outer housing configured to maintain the outer housing in an upright position.

3. The apparatus of claim 1, wherein the outer housing is constructed from a material selected from the group consisting of plastic, thin sheet metal, or 3D-printed polymer.

4. The apparatus of claim 1, wherein the modular base comprises an interchangeable mount including at least one of a pedestal, ground stake, and shepherd's hook attachment.

5. The apparatus of claim 2, wherein the solar lighting module comprises a solar panel, rechargeable battery, and LED circuit.

6. The apparatus of claim 1, further comprising a drainage port arranged at the base of the outer housing for water resistance.

7. The apparatus of claim 1, wherein the bottle is removable for independent handheld use.

8. The apparatus of claim 1, further comprising a fragrance insert positioned within the outer housing to emit scent during use.

9. The apparatus of claim 1, wherein the scrubber cap includes an integrated squeegee.

10. The apparatus of claim 1, wherein the bottle is refillable.

11. The apparatus of claim 1, wherein the inner housing contains additional openings, the solar lighting module being configured to project light through the additional openings to create illuminated visual effects.

12. A method of sanitizing outdoor surfaces using the apparatus of claim 1, comprising the steps of:
a. rotating the inner container housed within the outer housing to align the inner container opening with the corresponding outer housing access window;
b. removing or exposing the bottle;
c. dispensing a sanitizing fluid onto a target surface by actuating the squeeze bottle and scrubbing the target surface using the integrated cap to remove contaminants; and
d. placing the bottle back into the inner container opening and rotating the opening to secure the bottle within the outer housing.

13. The method of claim 12, and further comprising the step of mounting the apparatus on a surface including one of a mailbox post, patio deck, table surface, shepherd's hook, and ground stake.

14. The method of claim 13, and further comprising the step of illuminating an address display or decorative design on the outer housing using the integrated solar lighting.
